(19) United States
(12) Patent Application Publication
(10) Pub. No.: US 2014/0196721 A1
(43) Pub. Date: Jul. 17, 2014

(54) Pressure release means and placement indicator for inflatable catheter or respiratory gas supply retention means

(71) Applicant: Terence Gilhuly, Vancouver (CA)

(72) Inventor: Terence Gilhuly, Vancouver (CA)

(21) Appl. No.: 13/740,183

(22) Filed: Jan. 12, 2013

(51) Int. Cl. A61M 16/04; A61M 25/00

(52) U.S. Cl. CPC A61M 16/044; A61M 25/0017
USPC 128/207.15; 604/544

(57) ABSTRACT

Under- and overinflation of retention balloons and cuffs for catheters including Foley catheters, and for endotracheal tubes, tracheostomy tubes, laryngeal mask airways and similar devices is correlated with hospital acquired infections and with patient injury. In this patent, a solution that correctly inflates inflatable retention devices is taught, whereby a pressure release means is incorporated into the inflation pathway from the fluid input syringe to the retention means while inflating. This pressure release means can be set to open at or near the optimal fluid pressure so that overinflation can be prevented by releasing any pressure above the optimal pressure and underinflation can be prevented by a user pressurizing the cuff until the valve is noticed to open. As well, observation of fluid leaked through the pressure relief means can indicate misplacement of the retention means, allowing the user to reposition it before patient injury occurs. The invention is presented as a standalone part and in a format whereby the invention is integrated into existing airway cuff inflation designs.

### CROSS-REFERENCE TO RELATED APPLICATIONS

The present application claims priority from U.S. provisional patent application Ser. No. 61/586,460 filed Jan. 13, 2011.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

Endotracheal tubes (ETT), tracheostomy tubes, laryngeal mask airways (LMAs) and similar devices are employed to provide respiration to patients in hospital settings when undergoing procedures or recovering or otherwise are unable to breathe. A typical ETT—such as the Mallinckrodt Hi-Lo Cuffed Tracheal Tube—comprises a tube to act as an airway, a cuff to secure the airway (a “retention means”), an inflation line to that cuff, a pilot balloon to assess pressure in the cuff, and an injection port with a one-way non-return valve to allow injection of fluid—typically air, water or saline—into the cuff via the pilot balloon and inflation line.

For brevity, ETT will be used herein to refer to the mentioned endotracheal tubes (ETT), tracheostomy tubes, laryngeal mask airways (LMAs) and similar devices collectively, unless specific circumstances or implementations require otherwise. ETT is used herein as a general reference to “respiratory gas supply means entering the mouth or a tracheotomy incision” from the definition for Patent Classification class 128/207.14 including endotracheal tubes, tracheal tubes, tracheostomy tubes, breathing tubes, intubation tubes and LMAs. LMAs differ from the others in how they establish an airway as LMAs provide a supraglottic airway, however for the purposes of this invention the point of interest is the inflation means securing them while in use which is similar.

Physicians (most often anesthetists) and/or nurses need a safe means of securing the ETT to the patient so that respiration can be maintained reliably throughout the patient's procedure and stay on the ventilator, and until the ETT is intentionally removed.

ETTs are typically held in place with an inflatable cuff, inflated by injecting into it typically either air or saline. For endotracheal and tracheostomy tubes with inflatable cuffs, the cuff is inserted into the trachea and for an LMA the cuff is inserted into the pharynx. Typically, cuff inflation is done without measurement of the pressure inside the cuff. Frequently there will be a pilot bulb, which can be squeezed or looked at to get an idea of the internal pressure in the cuff, but this measurement is rarely quantified. Also as noted in Liu et al (Liu, Jianhui et al; “Correlations between controlled endotracheal tube cuff pressure and postprocedural complications: a multicenter study” Anesth Analg 2010; 111:1133-7), inflation done without measurement leads to wide variation in cuff pressures. Their work revealed an average cuff pressure of 43+/−23.3 mmHg, sometimes going as high as 210 mmHg.

If the cuff is underinflated there is a risk of the ETT slipping or falling out, and/or for fluids, such as saliva, to enter the trachea possibly leading to pneumonia and death. If the cuff is overinflated there is a risk of local tissue damage and further adverse events to the patient. Overinflation of endotracheal tube cuffs has been linked to the following adverse events: abrasion of the arytenoid cartilage vocal process; cartilage necrosis; cicatrix formation; consequences of failure to ventilate including death; damage to the perichondrium; development of dense or diffuse fibrosis invading the entire glottic area; emphysema; endobronchial aspiration; endobronchial intubation (hypoxemia); endotracheobronchial aspiration; epistaxis; esophageal intubation (stomach distention); excoriated membranes of the pharynx; eye trauma; fibrin deposition; formation of subglottic web; fracture-luxation of cervical column (spinal injury); fragmentation of cartilage; glottic edema (supraglottic, subglottic retroarytenoidal); granuloma of the inner arytenoid area; infections (laryngitis, sinusitis, abscess, respiratory tract infection); inflammation; intermittent aphonia and recurrent sore throat; laryngeal fibrosis; laryngeal granulomas and polyps; laryngeal obstruction; laryngeal stenosis; laryngeal ulcers; laryngotracheal membranes and webs; membraneous glottic congestion; membraneous tracheobronchitis; mild edema of the epiglottis; mucosal sloughing; paresis of the hypoglossal and/or lingual nerves; perforation of esophagus; perforation of the trachea; pneumothorax; replacement of the trachea wall with scar tissue; respiratory obstruction; retrobulbar hemorrhage; retropharyngeal abscess; retropharyngeal dissection; rupture of the trachea; sore throat; dysphagia; stricture of nostril; stridor; subglottic annular cicatricial stenosis; submucosal hemorrhage; submucous puncture of the larynx; superficial epithelial abrasion; swallowed tube; synechia of the vocal cords; teeth trauma; tissue burns; tracheal bleeding; tracheal stenosis; trauma to lips, tongue, pharynx, nose, trachea, glottis, palate, tonsil, etc.; traumatic lesions of the larynx and trachea; ulcerations exposing cartilaginous rings and minor erosions at cuff site; ulceration of the lips, mouth, pharynx; ulcers of the arytenoid; vocal cord congestion; vocal cord paralysis; and vocal cord ulcerations. [Source: Mallinckrodt Lo-Prof Tracheal Tube Cuffed Instructions for Use]

It is generally accepted that the correct inflation pressure is 25 cmH2O (18.4 mmHg or 0.36 psi) for ETTs. With a 20% tolerance for error, a range of acceptable pressures may be between 20 and 30 cmH2O.

A solution to the above problems of under- and overinflation of the ETT cuff, and the subject of this invention, is the incorporation of a pressure release valve into the inflation pathway from the fluid input syringe to the cuff while inflating. This valve can be set to open at or near the optimal fluid pressure so that overinflation can be prevented by releasing pressure above the optimal pressure and underinflation can be prevented by a user pressurizing the cuff until the valve is noticed to open. Valve incorporation into the inflation pathway could be in the form of an adaptor to use with standard commercially available ETTs, or as a valve integrated into a new design for ETTs.

A related problem exists for insertion of other catheters that require inflation of a retention means for fixation (temporary or otherwise) in a body space. If the insertion is correct but the inflation pressure is too low, the catheter may slip. If the insertion is placed incorrectly and the retention means is inflated in a location that is too small to accommodate the inflated size, tissue damage may occur. For example, over-inflating a balloon in a blood vessel could lead to rupture of the vessel. Also, consider Foley and other indwelling catheters for the administration and collection of fluids. Foley catheters can be used to collect urine for patients that are incontinent and for patients undergoing procedures in which they are unconscious. In this use, Foley catheters are inserted partially into the bladder, to a point where an inflatable retention means—typically a balloon—located near its distal tip can be inflated to prevent the catheter from being pulled out. Should the catheter not be inserted far enough and the balloon not enter the bladder, the balloon could be inflated within the patient's urethra resulting in pain if the patient is conscious, and if they are unconscious the overinflation may go unnoticed and the patient may also experience tissue damage due to the compression of the urethra. Foley catheters can also be used in gastrostomy, such as for a Janeway gastrostomy procedure where the catheter is used to provide access to the patient's stomach. Underinflation of the balloon for these catheters can lead to complications including loose catheters and spills.

Foley catheters comprise a tube to conduct fluids, an inflatable balloon to secure the catheter from inside the space it is to conduct fluids to and/or from (e.g. the bladder for urine collection), an inflation line to the balloon, (optionally) a pilot balloon to assess pressure in the balloon, and an injection port with a one-way non-return valve to allow injection of fluid—typically water or saline—into the balloon via the inflation line. The one-way non-return valve may be similar to those of the ETTs constructed with plungers and springs, or in a simple version they can be as simple as an elastomer cap over the interface to the inflation port through which a needle punctures when injecting.

Similar to the ETTs, a pressure release valve may be incorporated into the Foley catheter's inflation pathway from the fluid input source to the balloon to solve this problem of under- and overinflation of the Foley catheter balloon. The valve can be set to open at or near the optimal fluid pressure so that overinflation can be prevented by releasing pressure above the optimal pressure and underinflation can be prevented by a user pressurizing the cuff until the valve is noticed to open. Depending on what volume of fluid is pumped into the cuff before the valve opens, the physician can also determine whether or not the balloon cuff has been inflated where it was intended, such as the bladder, or in an undesirable location, such as the urethra.

For a catheter such as a Foley catheter, the adaptor implementation of the invention can work as an adaptor interposed between the Foley catheter balloon's inflation means and the Foley catheter balloon. In some cases the inflation port may be a Luer fitting with a check valve (one-way valve) as for the ETTs above and the use would be the same, with possible adaptation to correct for pressure differences required to maintain the balloon's retention under the pressures of the bladder. In other cases the catheter's inflation port may be through a rubber, elastomeric or other covering where a needle is usually required. In this case the adaptor could either have a needle built in or be adapted for the use of a needle at its interface to the inflation port.

Valves and related mechanisms have been described in ETTs and catheters before. However, previous mechanisms have been bulky and cumbersome, inefficient, complicated, disruptive of the user's routine practice, and costly to the point where these solutions are rarely seen and the problems of over- and underinflation still exist. Contrasting previous solutions, the invention disclosed herein is of a simple construction with only one additional part to the standard catheter and ETT design; it is non-obtrusive; it conforms to the user's expected routine of standard practice; and it is very inexpensive due to its simplicity.

### Patent Documents

See U.S. Pat. Nos. 3,642,005, 3,794,043, 3,854,484, 4,000,741, 4,116,201, 4,119,101, 4,147,170, 4,159,722, 4,248,222, 4,284,084, 4,361,107, 4,403,988, 4,501,273, 4,584,998, 4,598,707, 4,649,914, 4,653,539, 5,360,003, 6,530,898, 7,360,556, 7,673,653, 7,717,116 and 7,987,851, and applications 20110220116, 20110220118 and 20110220119.

### SUMMARY OF THE INVENTION

One form of the invention is as an adaptor that is used with commercially available ETTs and catheters whose proper function requires inflation through an inflation port. The invention can be a hollow chamber interposed between an inflation means (such as a syringe) and the ETT or catheter, with a release valve to allow passage of a fluid above a certain pressure, and with interfaces to allow connection to the appropriate devices used to inflate the ETT's or catheter's cuff and to the connector to the ETT or catheter cuff's inflation line. Typically these interfaces can include an interface to receive a syringe input and an output interface to mate with the particular ETT tube being used. For adaptors used in hospital settings, the interfaces will usually be Luer type fittings.

In another embodiment of the invention the valve can be integrated into the inflation port of a modified version of a commercially available ETT or catheter. In this embodiment of the invention, the ETT's or catheter's cuff inflating injection port can be augmented to further include a pressure release valve. This pressure release valve preferably vents outside of the ETT or catheter and is preferably positioned in the expanded injection port external to the airspace created by the cuff, the cuff inflation line and pilot balloon on the proximal to the user side of the one-way valve. The one-way valve is able to function as it does for the typical ETT. In summary, the invention provides an automatic pressure release means without interfering with the normal operation and function of the ETT.

Methods of using the invention for inflating cuffs and balloons without under- or over-inflation are described. As well, methods for determining when inflation of a catheter's balloon occurs outside of the desired body space and in a more confined space are also described.

### BRIEF DESCRIPTION OF THE DRAWINGS

FIG. 1 is a perspective view of the endotracheal tube adaptor apparatus;

FIG. 2 is a side view of the endotracheal tube adaptor apparatus;

FIG. 3 is a cutaway view of the endotracheal tube adaptor apparatus from the side;

FIG. 4 is a perspective view of the modified endotracheal tube apparatus including the invention;

FIG. 5 is a side view of the modified endotracheal tube apparatus including the invention; and

FIG. 6 is a cutaway view of the modified endotracheal tube apparatus from the side.

FIG. 7 shows views of standard LMA and ETT devices.

FIG. 8 shows Foley catheters properly placed in a male and female (modified from http://calder.med.miami.edu/pointis/indwelling.html) with reference to the anatomy.

### DETAILED DESCRIPTION OF THE INVENTION

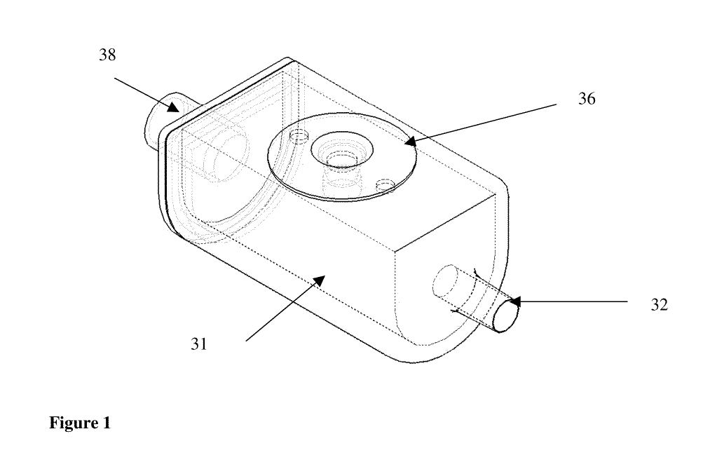

One embodiment of the invention as an adaptor to provide inflation pressure control to an ETT or other catheter with inflatable retention means will now be described with reference to FIGS. 1 to 3.

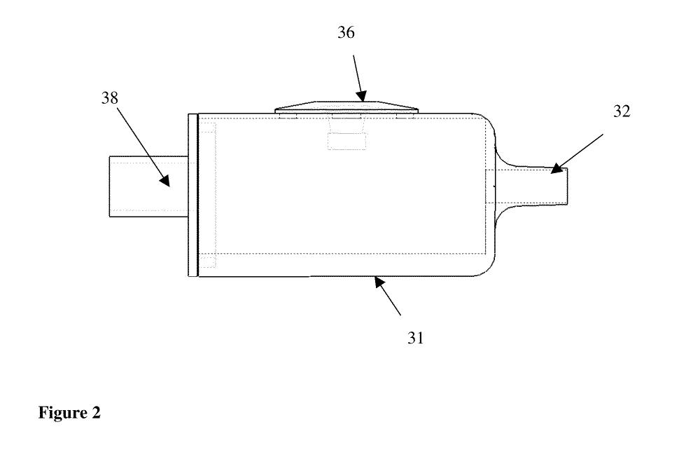

In FIG. 1, the adaptor is shown in a perspective view and in FIG. 2, the adaptor is shown in a side view. The adaptor as shown is comprised of a main body 31, valve 36 and fluid supply interface cap 38.

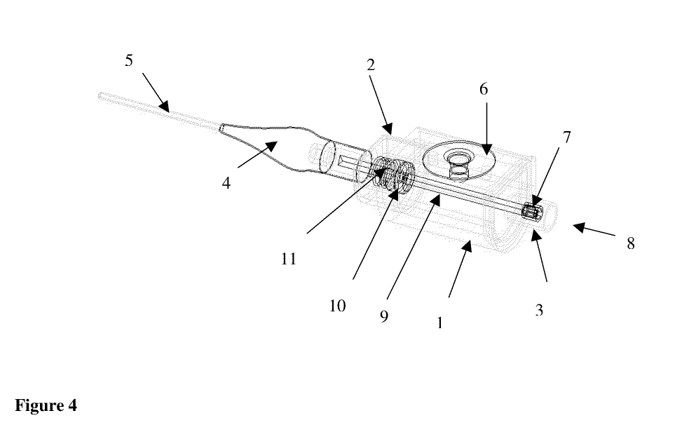

Main body 31 has distal and proximal ends with the distal end having the ETT or catheter interface 32 and with the proximal end connecting to the fluid supply interface cap 38. Main body 31 has a passage to allow fluid to move through main body 31 between and through ETT or catheter interface 32 and interface cap 38. Joined to main body 31 is valve 36, which in FIGS. 1 to 3 is drawn as an umbrella valve that joins to main body 31 preferably on an external surface. Main body 31 (and main body 1 in FIGS. 4 to 6) can be cheaply manufactured using plastics and can be dyed to have color and made opaque or transparent as desired. Candidate plastics include polycarbonate and Acrylonitrile butadiene styrene (ABS).

Fluid supply interface cap 38 provides an interface so that fluid may be delivered through the adaptor, into the ETT or catheter and to the ETT cuff or catheter balloon to inflate the cuff or balloon. As such, the fluid supply interface cap 38 can be designed to mate with a standard injection device, such as by having the geometry of the end of fluid supply interface cap 38 mate with a hospital syringe for hospital use.

Fluid supply interface cap 38 has been drawn in FIG. 1 as a separate part to main body 31 that is bonded to main body 31 but could be a part of main body 31 if the device was made with a process such as blow-molding.

ETT or catheter interface 32 is at the distal end of main body 31. ETT or catheter interface 32 functions to connect to the ETT or catheter tube to allow passage of the inflating fluid, and therefore ETT or catheter interface 32 preferably has geometry matching the ETT in use. These interfaces can conform to the aforementioned Luer standard, for example.

The valves in the figures (valve 36 and valve 136, for example) have been presented as umbrella valves but can be other valve types or mechanisms, such as rubber flaps, spring-loaded traps and duckbill valves.

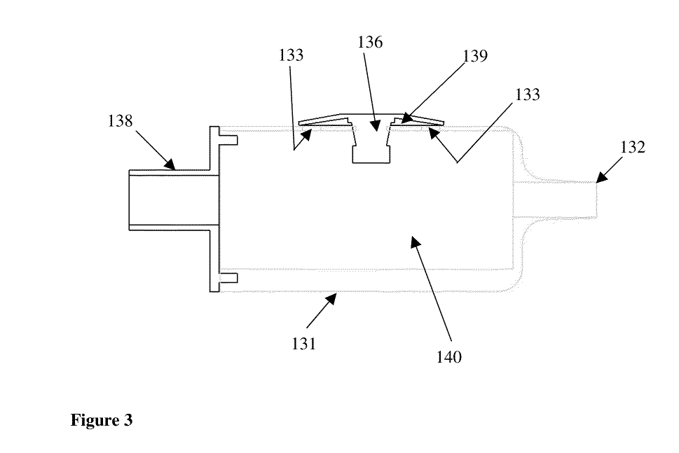

Valve 36 and valve 136 serve to cover fluid venting holes 133 (shown in FIG. 3) until the pressure inside main body 131 hollow space 140 and in air cavity 139 beneath valve 136 rises over a defined pressure, such as 25 cmH2O a recommended pressure for ETT cuffs to provide hold without causing injury to the patient. Valve 136 is designed to open at pressures above the defined pressure. In the case of an umbrella valve, the valve can be made of a flexible material (silicone, for example) such that the increased pressure causes valve 136 to deform and/or lift creating a gap through which fluid can leak.

In a typical use, a syringe (not shown) filled with fluid is connected to fluid supply interface cap 38. The syringe plunger (not shown) is pressed forcing air through fluid supply interface cap 38 and into main body 31. Air flows through ETT or catheter interface 32 and into the ETT or catheter (not shown) and the pressure rises in the cuff or balloon (not shown) inflating it. Simultaneously, the pressure increases in main body 31 and in the space between main body 31 and underneath valve 36 to match closely the pressure in main body 31. The user continues to force air or other fluid into the main body 31 and through it into the inflation port and cuff or balloon until the valve 36 opens indicating it has reached the specified pressure, or until the user is satisfied they have the appropriate pressure. If they have overfilled the cuff or balloon, the excess pressure will leak out through the valve.

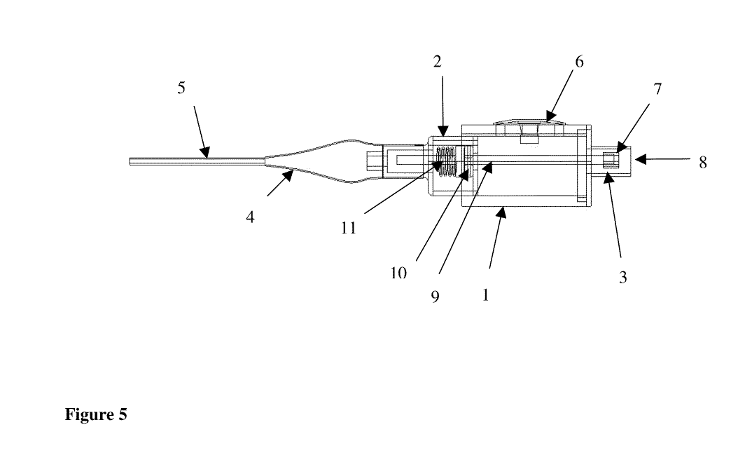

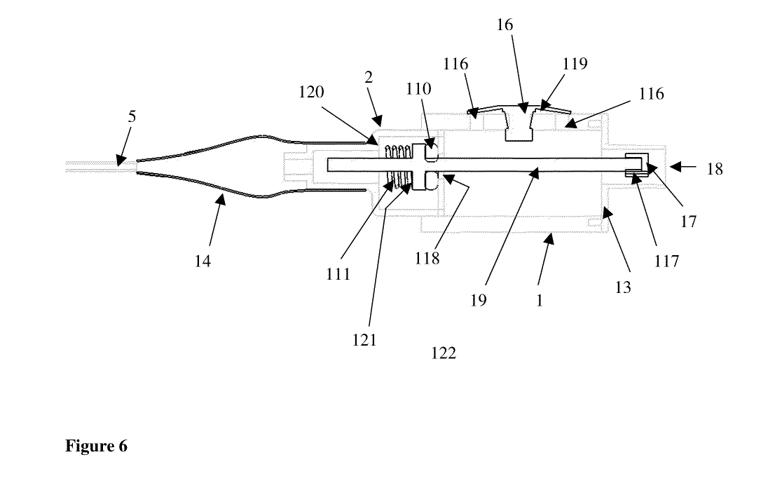

In another embodiment of the invention, the release valve function of the invention can be integrated into the inflation port of a standard ETT or catheter as shown in FIGS. 4 through 6. In FIG. 4, the integrated invention is shown in a perspective view and in FIG. 5, the integrated invention is shown in a side view. FIG. 6 shows a cutaway view to demonstrate the function of release valve 6 and the one-way non-return valve.

Similar to standard ETTs and catheters, an integrated embodiment of this invention has tubing 5 to conduct fluid to an inflatable cuff or balloon (not shown); a one-way valve to prevent pressure leaking out of the cuff or balloon comprised of a plunger 9 with plunger end cap 7, seal 10 and spring 11; and a main body 1 to contain the one-way valve and offer an interface for the inflating syringe to connect to. Optionally, the integrated embodiment of this invention can also have a pilot bulb 4 for indicating pressure level in the cuff or balloon by feel and sight. Main body 1 along with distal endpiece 2 and fluid supply interface endpiece 3 together are analogous to the injection port of the standard ETT. Distal endpiece 2 and fluid supply interface endpiece 3 have been drawn separately from main body 1 for ease of manufacture by injection molding, but could be combined.

Plunger 9 has been extended compared with the standard inflation port's plunger to accommodate the extra length of main body 1 which has been extended to accommodate valve 6 and allow pushing on plunger end cap 7 by the tip of a syringe delivering fluid through syringe interface 8.

Valve 6 has been added to prevent overinflation in the cuff or balloon and main body 1 has been modified to contain valve 6. Valve 6 has the same purpose and general function as valve 36 of the adaptor described above. When pressure in main body 1 and, due to the fluid connection between the two areas, in valve air cavity 119 beneath valve 16 in FIG. 6 rise above the selected pressure, valve 16 opens. With valve 16 open, inflation fluid can escape, releasing pressure until the internal pressure in main body 1 and the cuff or balloon reaches the defined pressure and valve 16 can close.

Looking at FIG. 6, operation of the one-way valve of the invention is seen to be similar to the standard ETT. When the cuff or balloon pressure is not being altered (the cuff or balloon is neither being inflated nor deflated), spring 111 is compressed such that it pushes against back wall 120 of distal endpiece 2 and the distal face of plunger spring rest 121 such that seal 110 is pressed against the back wall 122 of main body 1 preventing fluid from passing through main body 1 to end cap air passage 118.

In a typical use, when the user wishes to inflate the cuff or balloon, the user inserts a syringe into syringe interface 18 of fluid supply interface endpiece 13. The syringe contacts and displaces plunger end cap 17, moving plunger 19, further compressing spring 111 and forcing back plunger 19 and seal 110 to open air passage 118. The user may then press on the syringe to force fluid through main body 1, air passage 118, distal end cap 2, pilot bulb 14 and into the cuff or balloon (not shown) inflating it. Simultaneously, the pressure increases in main body 1 and in air cavity 119 beneath valve 16 to match closely the pressure in main body 31. Fluid venting holes 116 connect the air spaces inside main body 1 and air cavity 119. Again, once the pressure in the cuff and in main body 1 exceeds an optimal pressure, the pressure will also be increased in air cavity 119 causing valve 16 to open. Valve 16 will remain open until the pressure is reduced to the optimal level.

The user may deflate the cuff by inserting the syringe into syringe interface 18 to open the one-way valve as above, and then pulling back on the syringe to withdraw fluid into the syringe.

Plunger end cap 17 may further comprise channels 117 to prevent a seal forming between the syringe and the face of plunger end cap 17, which could limit the injection of fluid.

As with the previous instantiation's valve, valve 16 has been presented as an umbrella valve but can be other valve types or mechanisms, such as rubber flaps, spring-loaded traps and duckbill valves. Its role is to cover the fluid venting holes 116 (shown in FIG. 6) until the pressure inside main body 1 and in air cavity 119 beneath valve 16 rises over the defined pressure.

Valve 36 is preferably positioned proximal to the one-way valve (closer to the user and the inflation means) as shown in the figures, so that normal function of the one-way valve is preserved. Inflation, re-inflation and deflation procedures can be the same. Also, if there is accidental compression of the cuff such as by accidental contact with the patient, transmission of the increased pressure to the pressure release valve can be stopped by the one-way non-return valve and the cuff does not accidentally deflate.

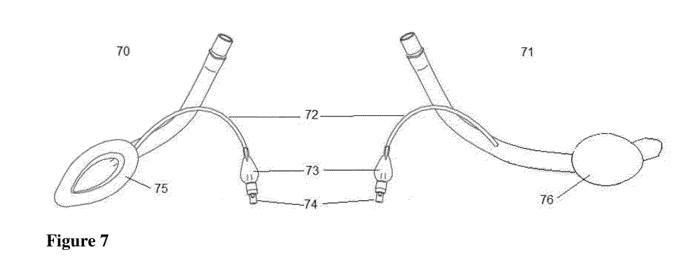

FIG. 7 has been added to show commercially available ETTs and their common parts as referenced in this invention disclosure. On the left is LMA 70 and on the right is endotracheal tube 71. The invention as an adaptor could interface to inflation port 74 to inflate pilot balloon 73 and through inflation line 72 either cuff 75 for LMA 70 or cuff 76 for endotracheal tube 71. The invention as an integrated part could be built into a modified version of inflation port 74.

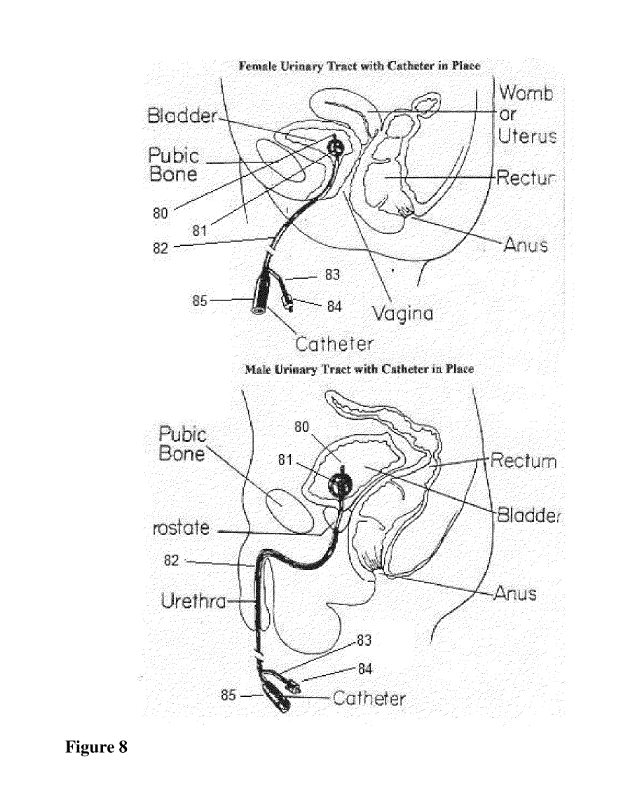

FIG. 8 has been added to demonstrate Foley catheters in use inside male and female patients and to illustrate how inflation outside of the bladder could be painful and damaging to the patient. The Foley catheter 82 comprises a tip 80 where fluid is dispensed and taken in (in this example of the figure, urine is removed), a balloon 81 for retention within the body space, inflation line 82 for directing fluid to balloon 81, inflation port 84 and fluid administration and/or collection port 85. In the diagram the balloon 81 is shown inflated and inside the bladder. The Foley catheters illustrated are standard devices not incorporating the invention. The invention could be used as an adaptor interfacing to inflation port 84, or the invention could be integrated into inflation port 84.

The invention can also be used to assess whether or not an ETT or catheter, particularly one with an inflatable retention means like the Foley catheter, has been inserted correctly. The user could proceed as they normally do for site preparation and insertion of the ETT or catheter. Once the ETT or catheter is in a position that the user things is correct, the user can attempt to inflate the cuff or balloon with a small amount of fluid less than the amount required to inflate the cuff or balloon under normal circumstances. For example, for a balloon requiring 10 mL to fill, the user could inject 3 mL and examine the valve. If at this point the valve is noted to have opened and/or have leaked fluid, the user can hypothesize that the cuff or balloon is in a position where it cannot expand freely and therefore should be reinserted and/or the patient's situation should be investigated further in case there is another issue beyond a short insertion. If there is no leakage of fluid, it can be assumed that the cuff or balloon is in the correct location and the full amount of fluid required can be injected.

What is claimed is:

1. An adaptor to connect the inflatable retention means of a catheter or a respiratory gas supply means to an inflation mechanism, where said catheter or respiratory gas supply retention means comprises a retention means to secure the catheter including an inflatable cuff or balloon, an inflation line to said retention means and an inflation port with a one-way valve through which an inflation fluid is injected to enter said inflation line and to fill said inflation line and said retention means, where said adaptor comprises a main body with proximal and distal ends and containing a cavity connecting the proximal and distal ends, and a pressure release means set to open when the internal pressure of said main body cavity reaches a defined pressure, where said proximal end has a connection means to receive an inflation means such as a syringe; and said distal end has a connection means to connect with said inflation port of said catheter or respiratory gas supply retention means.

2. The adaptor of claim 1 where said catheter or respiratory gas supply means includes one of a Foley catheter, an endotracheal tube, a tracheostomy tube or a laryngeal mask airway.

3. The adaptor of claim 1 where the pressure release means is one of an umbrella valve, a rubber flap, a spring-loaded trap and a duckbill valve.

4. The adaptor of claim 1 where the pressure release means opens at or above a pressure of 20 cmH2O.

5. A method of inflating a catheter or respiratory gas supply retention means to prevent over-inflation and under-inflation where said catheter or respiratory gas supply retention means comprises an inflatable retention means to secure the catheter including a cuff or balloon, an inflation line to said retention means and an injection port with a one-way valve through which an inflation fluid is injected to enter said inflation line and to fill said line and said retention means, including the steps:
a) the catheter or respiratory gas supply means is inserted into a patient;
b) a syringe containing a fluid including air, water or saline is connected to an adaptor, said adaptor comprising a main body with proximal and distal ends and containing a cavity connecting the proximal and distal ends, and a pressure release means set to open when the internal pressure of said main body cavity reaches a defined pressure, where
i. said proximal end has a connection means to receive an inflation means such as a syringe; and
ii. said distal end has a connection means to connect with said inflation port of said catheter or respiratory gas supply retention means
c) said adaptor is connected to said inflation port of catheter or respiratory gas supply retention means
d) said syringe is compressed to inject said fluid into said adaptor and into said inflation cuff; and
e) said compression of said syringe continues until said pressure release means is activated causing said pressure release means to open with or without fluid escaping through said pressure release means or said cuff is judged to be properly inflated.

6. The method of claim 5 where said catheter or respiratory gas supply means includes one of a Foley catheter, an endotracheal tube, a tracheostomy tube or a laryngeal mask airway.

7. The method of claim 5 where said pressure release means is one of an umbrella valve, a rubber flap, a spring-loaded trap and a duckbill valve.

8. The method of claim 5 where the pressure release means opens at or above 20 cmH2O.

9. A catheter or respiratory gas supply retention means comprising:
i. an inflatable retention means to secure the catheter including an inflatable cuff or balloon;
ii. an inflation line to said inflatable retention means;
iii. an injection port with a proximal end having an interface to accept input of a fluid, a one-way non-return valve, and a distal end in fluid connection with said inflation line, to allow input of fluid to inflate said retention means including inflatable cuff or balloon; and
iv. a pressure release means to prevent inflation of said inflatable retention means beyond a defined pressure.

10. The catheter or respiratory gas supply means of claim 9 where said injection port further comprises said pressure release means.

11. The catheter or respiratory gas supply means of claim 10 where said pressure release means is located outside of the fluid cavity defined by said cuff, inflation line and non-return valve.

12. The catheter or respiratory gas supply means of claim 9 where the pressure release means is one of an umbrella valve, a rubber flap, a spring-loaded trap and a duckbill valve.

13. The catheter or respiratory gas supply means of claim 9 where the pressure release means opens at or above a pressure of 20 cmH2O.

14. The catheter or respiratory gas supply means of claim 9 where said catheter or respiratory gas supply means includes one of a Foley catheter, an endotracheal tube, a tracheostomy tube or a laryngeal mask airway.

15. A method of preventing overinflation of an inflatable catheter or respiratory gas supply retention means when applying a catheter or respiratory gas supply to a patient, a catheter or respiratory gas supply retention means including said inflatable catheter or respiratory gas supply retention means comprising:
i. an inflatable retention means to secure the catheter including an inflatable cuff or balloon;
ii. an inflation line to said inflatable retention means;
iii. an injection port with a proximal end including an interface to accept input of a fluid, a one-way non-return valve, and a distal end in fluid connection with said inflation line, to allow input of fluid to inflate said retention means including inflatable cuff or balloon; and
iv. a pressure release means to prevent inflation of said inflatable retention means beyond a defined pressure.
including the steps:
a) The catheter or respiratory gas supply means is inserted into said patient;
b) a syringe containing a fluid is connected to said injection port;
c) said syringe is compressed to cause said fluid to inject into said adaptor and into said inflation cuff; and
d) said compression of said syringe continues until said pressure release means is activated or the user is satisfied said cuff is properly inflated.

16. The method of claim 15 where said pressure release means is one of an umbrella valve, a rubber flap, a spring-loaded traps and a duckbill valve.

17. The method of claim 15 where the pressure release means opens at or above 20 cmH2O.

18. The method of claim 15 where said catheter or respiratory gas supply means includes one of a Foley catheter, an endotracheal tube, a tracheostomy tube or a laryngeal mask airway.

19. A method for assessing proper insertion into a space in a body by a user, of a catheter or respiratory gas supply means including an inflatable retention means, an inflation line connecting to said inflatable retention means and an inflation port, said inflation port comprising a proximal end including an interface to accept input of a fluid, a one-way non-return valve, and a distal end in fluid connection with said inflation line, to allow input of fluid to inflate said retention means including inflatable cuff or balloon; and a pressure release means to prevent inflation of said inflatable retention means beyond a defined pressure, including the steps:
a) the user inserts said catheter or respiratory gas supply means;
b) the user attempts to inflate retention means with fluid injection source, injecting a known amount of fluid, less than an amount required to adequately fill the retention means;
c) the user observes said pressure release means while inflating;
d) should the user observe fluid to leak out or said pressure release means to open, the catheter or respiratory gas supply means is judged not to have been positioned properly and said inflatable retention means is deflated, said catheter or respiratory gas supply means is repositioned and the steps are repeated.

20. The method of claim 19 where said catheter or respiratory gas supply means includes one of a Foley catheter, an endotracheal tube, a tracheostomy tube or a laryngeal mask airway.
